(19) United States
(12) Patent Application Publication
(10) Pub. No.: US 2010/0280052 A1
(43) Pub. Date: Nov. 4, 2010

(54) USE OF ANTIVIRALS TO TREAT CMV-RELATED CONDITIONS

(76) Inventor: Paul Austin Henry Moss, Birmingham (GB)

Correspondence Address: BERLINER & ASSOCIATES, 555 WEST FIFTH STREET, 31ST FLOOR, LOS ANGELES, CA, 90013, US

(21) Appl. No.: 12/600,983

(22) PCT Filed: May 30, 2008
(86) PCT No.: PCT/GB2008/001844

§ 371 Date: Jun. 29, 2010

(30) Foreign Application Priority Data
May 30, 2007 (GB) 0710277.5

(51) Int. Cl. A61K 31/522; A61K 31/52; A61P 37/04; A61P 31/22; A61P 31/18; A61P 25/00; A61P 29/00; A61P 1/16; A61P 11/00; A61P 9/00; A61P 9/10; A61P 33/06; A61P 33/02

(52) U.S. Cl. 514/263.38; 514/263.4

(57) ABSTRACT

The invention provides for the use of compounds active against cytomegalovirus (CMV) in the preparation of medicaments for improving the immune response of a CMV-seropositive, immunocompetent individual, or for the amelioration of certain other medical conditions. Suitable compounds include the nucleoside analogues acyclovir, famciclovir, and valacyclovir. Infection with cytomegalovirus is widespread and commonly believed to be both asymptomatic in immunocompetent individuals and unbeatable without the use of highly cytotoxic drugs. It is suggested herein that, in fact, CMV infection produces a disproportionately large immune response, thereby weakening the ability of the immune system to respond to other infections (and hence is not asymptomatic). Further, treatment with comparatively low doses of drugs having low cytotoxicity (and hence similarly low efficacy) can reduce the magnitude of this CMV-specific immune response, improving the overall immune response, and ameliorating the symptoms of other medical conditions.

This application relates to the treatment of medical disorders or improvement of immune response. More specifically, it relates to use of an antiviral compound in the preparation of a medicament for the treatment of various medical disorders or improvement of immune response.

Existing medical treatments for a particular disorder will typically attempt to remove the cause of that disorder though physical and/or chemical means. For example, treatment of an abscess caused by bacterial infection might involve draining of the abscess together with the use of antibiotics. In general, medical treatments are limited to specific medical conditions, and may be weakened by factors such as drug resistance in pathogens. A great deal of research effort is therefore spent on developing new treatments for conditions previously thought to be untreatable, to improve recovery rates, or to overcome drug resistance or other changes in requirements.

The body's natural immune system is able to deal with a range of pathogens, foreign bodies, etc which would otherwise lead to medical disorders. The methods employed by the immune system are typically divided into the innate response (which includes inflammation, binding of complement proteins to foreign cells, and attack of cells by phagocytes, and is able to react quickly to a new type of infection) and the adaptive response (including antibodies and T cells) which target specific antigens. Although relatively slow to react to a new infection, the adaptive system has great specificity and is also able to build up an immunological memory, allowing a rapid response to a subsequent re-infection by the same pathogen.

Certain medical conditions are recognised as involving partial or complete inactivation of the immune response. For example, the immune response may sometimes be suppressed deliberately, such as in the case of transplant patients to prevent rejection of the transplant. Such suppression is commonly achieved by administration of glucocorticoids, cytostatic agents, or other immunosuppressant drugs. Many conditions such as AIDS (caused by HIV infection), certain malignant diseases, drug abuse and malnutrition also cause unwanted immunodeficiency. In both cases, the loss of immune function can lead to serious illness, and so further action must be taken to prevent this. Where the immune response has been deliberately suppressed, then potential sources of infection must be targeted directly, such as by prophylactic administration of antibiotics. In situations where the immunodeficiency is not a prerequisite of a medical procedure, for example in severe dietary deficiency or advanced HIV infection, then it is appropriate to attempt to augment immune function through the use of agents such as caloric augmentation or anti-retroviral treatment.

However, it is also desirable to optimise the activity of the immune response, and thereby ameliorate the symptoms of other illnesses, in patients (or apparently healthy individuals) in whom immune function is not significantly depressed but in whom the immune system is not operating at an optimal level. These conditions are not considered as immunodeficient in the conventional sense and patients with this disorder would not be susceptible to the typical opportunistic infections that are associated with substantial immunosuppression. Thus these individuals are generally considered as immunocompetent and the potential advantage of optimising their immune function has not previously been appreciated. The present invention has been conceived to at least in part address this issue. For the purposes of the present invention such individuals are therefore termed hereafter as immunocompetent.

According to a first aspect of the present invention, there is provided the use of a compound effective against cytomegalovirus (CMV) in the preparation of a medicament for improving the immune response of a CMV-seropositive, immunocompetent individual.

As used herein, the phrase ‘effective against CMV’ is intended to mean that the ID₅₀ of the compound against CMV is less than 1000 μM.

As used herein, ‘improving the immune response’ is intended to mean reducing the number of effector and/or memory T cells that are specific for cytomegalovirus, and/or increasing the number of naïve T cells. In one embodiment, at least one of the reduction (in CMV-specific effector and/or memory T cells) and increase (in naïve T cells) is at least 20%. In a further embodiment, at least one of the reduction and increase is at least 40%. In a still further embodiment, at least one of the reduction and increase is at least 60%. It is believed that these effector and memory T cells specific for CMV contribute to reduction in immune function by taking up excessive resources of the lymphoid system, by suppressing the number of naïve T cells and by secreting soluble factors in the blood stream and lymphoid system.

As used herein, ‘seropositive’ has the ordinary meaning of indicating the detectable presence of antibodies specific for CMV in the blood, and indicates a history of past infection.

In one embodiment the number of CMV-specific memory or effector T cells is taken to be represented by the number of CD4+ T cells which have lost expression of the CD28 molecule on their surface. These CD4+ CD28-T cells are a characteristic feature of the CMV-specific CD4+ T cell response according to the publication of van Leeuwen et al, J. Immunology, 2004. As such, the proportion or number of these cells in peripheral blood is taken to represent a valuable way to determine the CMV-specific immune response. This value may be used to determine the individuals in whom the size of the CMV-specific immune response, indicates that they are particularly likely to benefit from the treatment. In addition this measurement may be used to monitor the response to treatment and therefore to guide the efficacy of response and the potential need to modify the treatment dose.

In one embodiment the number of CMV-specific memory or effector T cells is taken to be represented by the number of CD4+ T cells which have lost expression of the CD27 molecule on their surface. CD4+ CD27-T cells are a characteristic feature of the CMV-specific CD4+ T cell response according to the publication of Pourgheysari et al, J. Virology, 2007. As such, the proportion or number of these cells in peripheral blood is taken to represent a valuable way to determine the CMV-specific immune response. This value may be used to determine the individuals in whom the size of the CMV-specific immune response indicates that they are particularly likely to benefit from the treatment. In addition this measurement may be used to monitor the response to treatment and therefore to guide the efficacy of response and the potential need to modify the treatment dose.

In one embodiment the number of CMV-specific memory or effector T cells is taken to be represented by the number of CD4+ T cells which have gained expression of the CD57 molecule on their surface. These CD4+ CD57+ T cells are a characteristic feature of the CMV-specific CD4+ T cell response according to the publication of Pourgheysari et al, J. Virology, 2007. As such, the proportion or number of these cells in peripheral blood is taken to represent a valuable way to determine the CMV-specific immune response. This value may be used to determine the individuals in whom the size of the CMV-specific immune response indicates that they are particularly likely to benefit from the treatment. In addition this measurement may be used to monitor the response to treatment and therefore to guide the efficacy of response and the potential need to modify the treatment dose.

It will be understood that the number of CMV-specific memory or effector T cells may be measured by the pattern of expression of any combination of CD27, CD28 or CD57 on the surface of CD4+ T cells. Alternatively or in addition, the number of CMV-specific memory or effector T cells may be measured by the pattern of expression of any combination of CD27, CD28 or CD57 on the surface of CD8+ T cells. The proportion or number of CD8+ cells in peripheral blood that have lost expression of CD27 or CD28 is increased in individuals who are CMV seropositive. The number of CD8+ T cells that express the CD57 molecule is increased in CMV seropositive individuals.

As used herein, ID₅₀ values are measured as defined in the assay of Crumpacker et al., Growth inhibition by acyloguanosine of herpesviruses isolated from human infections, Antimicrobial Agents and Chemotherapy, 1979, volume 15, number 5, pages 642-645.

In one embodiment, the medicament is for improving the immune response of a CMV-seropositive, immunocompetent individual suffering from at least one condition listed in the second aspect of the invention.

In one embodiment, improving the immune response comprises reducing the CMV-specific immune response, and thereby improving overall immune response.

According to a second aspect of the invention, there is provided the use of a compound effective against CMV in the preparation of a medicament for treatment of CMV in an immunocompetent individual.

As used herein, ‘treatment of CMV’ is intended to mean the amelioration of one of more effects of CMV infection, and should not be taken to require the complete removal of CMV from an infected individual. In one embodiment, treatment of CMV may be represented by a reduction in the proportion of effector and/or memory T cells that are specific for cytomegalovirus. As above, the number of CMV-specific memory or effector T cells may be represented by the number of CD4+ or CD8+ T cells which have lost expression of CD28 or CD27, or gained expression of CD57, or any combination of these.

According to a third aspect of the present invention, there is provided the use of a compound effective against CMV in the preparation of a medicament for amelioration of the symptoms of a condition selected from: HIV infection; mood disorders including depression, fatigue or anxiety; schizophrenia; chronic fatigue syndrome; inflammatory conditions such as rheumatoid arthritis, systemic lupus erythematosus, sarcoidosis, ankylosing spondylitis, psoriatic arthropathy, Wegener's granulomatosis, and other vasculitidies; autoimmune conditions such as multiple sclerosis, Sjorgen's syndrome, diabetes, primary biliary sclerosis, and systemic sclerosis; malignant disease; sarcoidosis or amyloidosis; chronic bacterial, viral or parasitic infection including viral hepatitis and Chagas' disease; malaria; chronic lung disease such as bronchiectasis or cystic fibrosis; acute coronary syndromes such as unstable angina; arterial (e.g. aortic) aneurysm; atherosclerosis; and cerebrovascular occlusion (‘stroke’).

According to a fourth aspect of the present invention, there is provided the use of a nucleoside analogue having an ID₅₀ against CMV of greater than 20 μM (as defined in the assay of Crumpacker et al) in the preparation of a medicament for the treatment of CMV-mediated disorders in immunocompetent patients.

In one embodiment, the nucleoside analogue has an ID₅₀ against CMV of at least 50 μM. In a further embodiment, the nucleoside analogue has an ID₅₀ against CMV of at least 100 μM.

In one embodiment, the nucleoside analogue has an ID₅₀ against CMV of less than 1000 μM.

According to a fifth aspect of the present invention, there is provided a method of medical treatment comprising administering to a CMV-seropositive immunocompetent patient a compound effective against CMV to reduce the immune response against CMV.

The following points are applicable to all aspects of the present invention.

In a preferred embodiment, the compound or nucleoside analogue is selected from acyclovir, famciclovir and valacyclovir.

Cytomegalovirus (CMV) is a human herpes virus, one of the family of Herpesviridae. Other human herpes viruses cause diseases such as oral and/or genital herpes (herpes simplex viruses), chicken pox and shingles (varicella zoster virus), and Burkitt's lymphoma (Epstein-Barr virus).

CMV infection is extremely widespread and is generally considered to be asymptomatic in immunocompetent individuals. Infection rates (based on the detectable presence of antibodies in the body) vary according to geographical region: in Africa the vast majority of the population is infected by the age of five years; in Japan the majority of the population is infected by the age of twenty years; whilst in Europe and North America approximately 70% of people are infected by the age of sixty years.

After primary infection, which may occur at any age, CMV typically remains latent in immunocompetent people; this is believed to be a result of the action of the CMV-specific immune response, which is generally able to limit lytic viral replication. Although the exact details are unclear at present, it is believed that the virus may enter a ‘latent’ state following primary infection but undergoes episodes of reactivation during which the immune response is critical to control widespread viral dissemination.

The fact that this reactivation may be a frequent occurrence is revealed by the fact that patients who receive intense immune suppressive treatments often suffer from clinical CMV viral reactivation within a few weeks. CMV reactivation, and the tissue damage arising from this, are well recognised problems in heavily immunosuppressed patients such as those who have received an allogeneic transplant or have advanced HIV infection. In such cases, CMV is known to cause diseases such as pneumonitis, colitis, and retinitis.

During periods in which the CMV infection is latent, the infected person remains seropositive, and the medicaments and methods of treatment described herein remain applicable.

The present invention arises from the discovery that, rather than being asymptomatic as previously thought, CMV infection in immunocompetent people in fact contributes to the severity of certain medical conditions. These can be broadly divided into two classes (collectively referred to herein as CMV-mediated disorders):

1) Impairment of Immune Function Secondary to CMV Infection

Infection with CMV can produce modification and impairment of the natural immune function in consequence of development of a CMV-specific immune response. This can take various forms, including:
reduced immune function, particularly at times of physiological or psychological stress;
increased development of immune senescence, as shown for example by accumulation of memory T cells, or accelerated loss of naïve T cells;
reduced vaccine response to other immunogens.

This is particularly relevant to those in whom the immune system is already compromised or at risk of compromise, such as:
elderly individuals;
patients undergoing renal dialysis;
patients at risk of malarial infection;
patients who have received an allograft;
patients who have undergone stenting or surgical bypass of coronary arteries;
prevention of cerebrovascular occlusion (‘stroke’) in patients with identifiable risk factors;
individuals undergoing space exploration, such as astronauts.

2) Increase in the Severity of Symptoms of other Medical Disorders

These can occur in a number of medical conditions, including:
HIV infection at any stage of the disease;
mood disorders including depression, fatigue or anxiety;
schizophrenia;
chronic fatigue syndrome;
inflammatory conditions such as rheumatoid arthritis, systemic lupus erythematosus, sarcoidosis, ankylosing spondylitis, psoriatic arthropathy, Wegener's granulomatosis and other vasculitidies;
auto-immune conditions such as multiple sclerosis, Sjögrens's syndrome, diabetes (including complications such as erectile dysfunction), primary biliary sclerosis and systemic sclerosis;
malignant disease;
sarcoidosis or amyloidosis;
chronic bacterial, viral or parasitic infection, such as viral hepatitis or Chagas' disease;
malaria;
chronic lung disease such as bronchiectasis or cystic fibrosis;
acute coronary syndromes such as unstable angina;
arterial (e.g. aortic) aneurysm;
atherosclerosis;
cerebrovascular occlusion (strokes);
Alzheimer's disease;
large granular lymphocytosis;
Guillan-Barre syndrome;
inflammatory bowel disease such as Crohn's disease or ulcerative colitis;
clinical disorders secondary to alcohol intake.

Without wishing to be bound by theory, it is believed that the medical disorders listed above are exacerbated in a CMV-infected patient. This is thought to be related to the development of clinical sequelae resulting from the immune modulatory effect of the disproportionately large immune response produced by CMV infection. It is therefore possible to improve treatment of the medical conditions by reducing this immune response through the use of an antiviral drug, without requiring eradication of the infection.

The evidence that cytomegalovirus infection can exacerbate the clinical symptoms of these diseases results from two observations. The conditions listed above are associated either with an increased prevalence of CMV seropositivity (compared to control groups) or are often associated with elevated levels of CD28-T cells which may be taken to represent a heightened CMV-specific immune response. The immune response against CMV is therefore amplified in patients with these conditions, and is likely to reflect and/or contribute to a state of mild immunosuppression or immune dysregulation that can be at least partially reversed by aspects of the present invention.

Certain antiviral drugs are known for the treatment of herpes virus infections. In particular, nucleoside analogues such as acyclovir (also known as aciclovir) (9-(2-hydroxyethoxymethyl)guanine-GB 1 523 865) are known for the treatment of human herpes viruses, being most effective against herpes simplex viruses (HSV-1 and HSV-2) and varicella zoster virus (VZV). Acyclovir has minimal activity against CMV replication in vitro, with an ID₅₀ (the concentration at which the drug reduces viral plaque formation by 50% or more) of greater than 100 μM, compared to values of 0.15 μM, 1.62 μM and 3.75 μM against HSV-1, HSV-2 and VZV respectively (according to the assays of Crumpacker et al.). Valacyclovir is the L-valine ester of, and acts as a prodrug for, acyclovir, and hence has the same mechanism of action. Both drugs produce a number of side effects at common doses, including nausea, vomiting, diarrhoea and/or headaches.

Ganciclovir and valganciclovir are homologues of acyclovir and valatyclovir respectively, which have been developed for their markedly improved activity against CMV. These drugs are widely used in the management of CMV disease in immunosuppressed patients. However, ganciclovir is considered a potential human carcinogen, teratogen, and mutagen, and is considered likely to cause inhibition of spermatogenesis. Known side-effects include granulocytopenia, neutropenia, anaemia, thrombocytopenia, fever, nausea, vomiting, dyspepsia, diarrhoea, abdominal pain, flatulence, anorexia, raised liver enzymes, headache, confusion, hallucination, seizures, pain and phlebitis at injection site (due to high pH), sweating, rash, itch and increased serum creatinine and blood urea concentrations.

Penciclovir is an analogue of acyclovir which is able to treat herpesvirus infections with fewer side effects. The antiviral spectrum of penciclovir is similar to that of acyclovir, and it therefore has minimal activity against CMV. Famciclovir is a prodrug for penciclovir, having improved bioavailability.

There is therefore a prejudice in the art against treatment of CMV in immunocompetent patients, based on the following beliefs:
most antiviral drugs are not able to effectively treat CMV;
those drugs that are able to treat CMV have unacceptably high toxicity and level of side-effects;
CMV infection is near-ubiquitous and has been considered to be asymptomatic in immunocompetent patients.

However, the inventors have discovered that CMV infection is not entirely asymptomatic, and that surprisingly there are in fact benefits in treating CMV infected patients with antiviral drugs which have low activity against cytomegalovirus as estimated by in vitro assays. When given to patients these drugs cause a substantial reduction in the immune response to cytomegalovirus, most likely by their ability to reduce subclinical episodes of CMV reactivation and hence limit recurrent stimulation of the CMV-specific immune response.

The dosage administered to a patient will normally be determined by the prescribing physician and will generally vary according to the age, weight and response of the individual patient, as well as the severity of the patient's symptoms. However, in most instances an effective therapeutic daily dosage will be in the range of 200-2000 mg and, preferably, of 400-1000 mg (e.g. for valacyclovir or acyclovir) administered in single or divided doses. In some cases, however, it may be necessary to use dosages outside these limits.

While it is possible for an active ingredient to be administered alone as the raw chemical, it is preferable to present it as a pharmaceutical formulation. The formulations, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

For the avoidance of doubt, the activity of the active ingredient of the present invention may be affected by other ingredients of a pharinaceutical formulation. The active ingredient may form a ‘combination therapy’ with any other ingredient of the pharmaceutical formulation. For example, one possible such combination therapy involves the use of zinc in one of the formulations of any aspect of the present invention. Alternative combination therapies may include the use of ‘statin’ drugs such as simvastatin in one of the formulations of the present invention, or the inclusion of anti-tumour necrosis factor (TNF) drugs such as infliximab in formulations for use in the management of musculo-skeletal disease.

Conveniently, unit doses of a formulation contain between 200 mg and 800 mg of the active ingredient. Preferably, the formulation is suitable for administration from one to six, such as two to four, times per day. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, nasal or buccal administration.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200 μm.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

Formulations of the present invention may also be in the form of a self-propelling formulation wherein the active ingredient is present in solution.

Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, ie by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

The invention will now be described by way of example only with reference to the accompanying figures, in which:

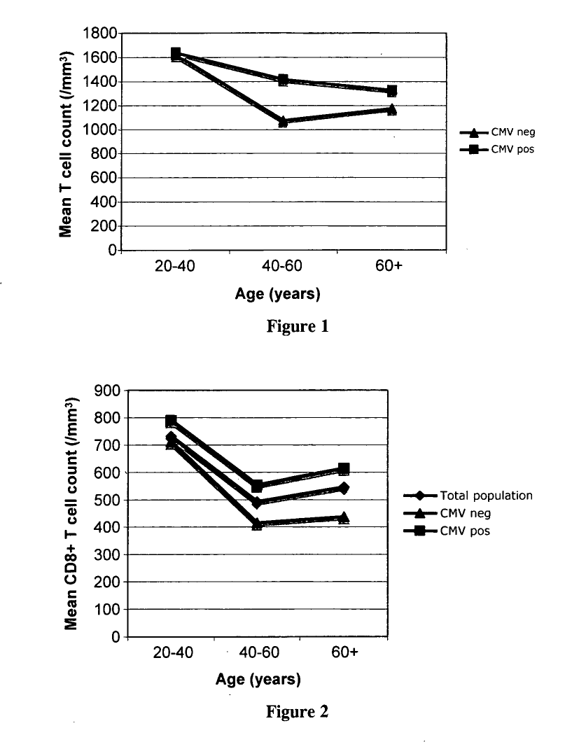

FIG. 1 shows the effect of CMV infection on T cell counts in peripheral blood;

FIG. 2 shows the effect of CMV infection on CD8+ T cell counts in peripheral blood;

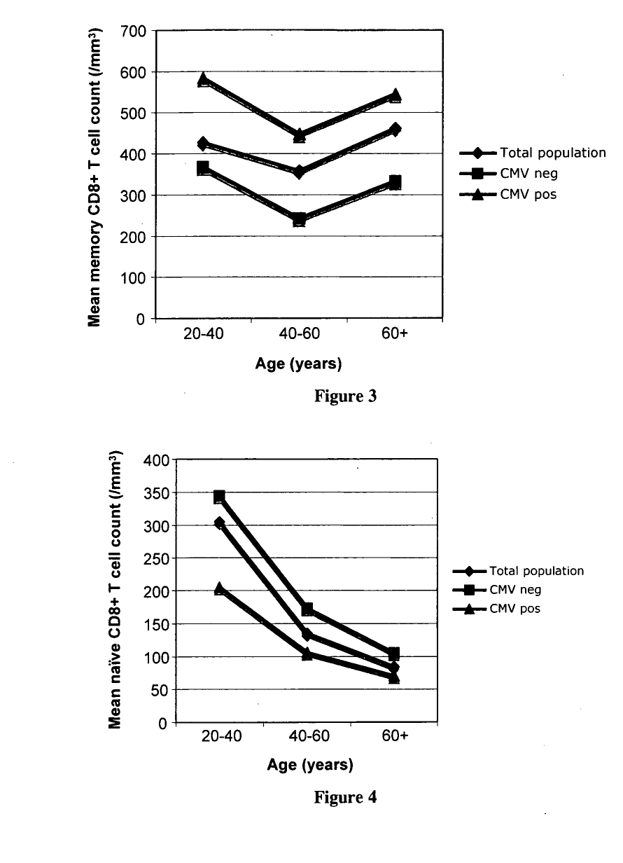

FIG. 3 shows the effect of CMV infection on memory and effector CD8+ T cell counts in peripheral blood;

FIG. 4 shows the effect of CMV infection on naïve CD8+ T cell counts in peripheral blood;

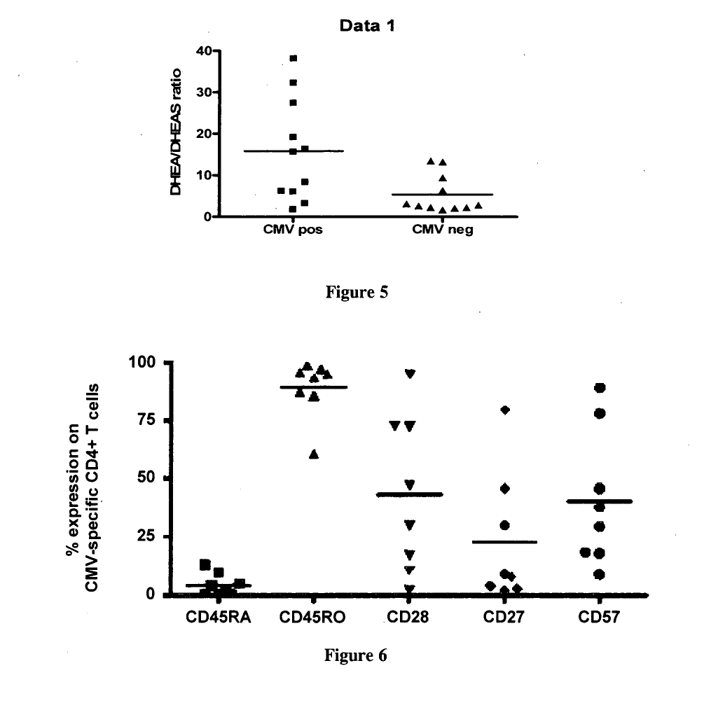

FIG. 5 shows the effect of CMV infection on the blood ratio of dehydroepiandrosterone (DHEA) and the corresponding sulfate (DHEAS);

FIG. 6 shows the phenotype of CMV-specific CD4+ T cells which were revealed by stimulating peripheral blood from eight healthy donors with a lysate of CMV-infected cells;

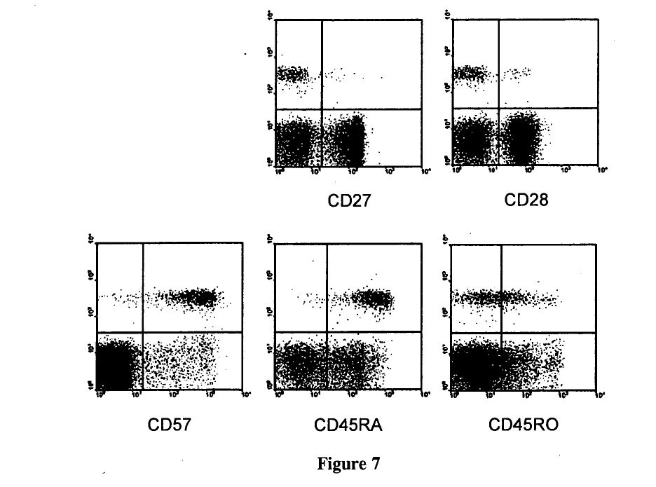

FIG. 7 shows the phenotype of CMV-specific CD8+ T cells which were produced by staining peripheral blood from a healthy donor with an HLA-peptide tetramer containing an immunodominant peptide from CMV;

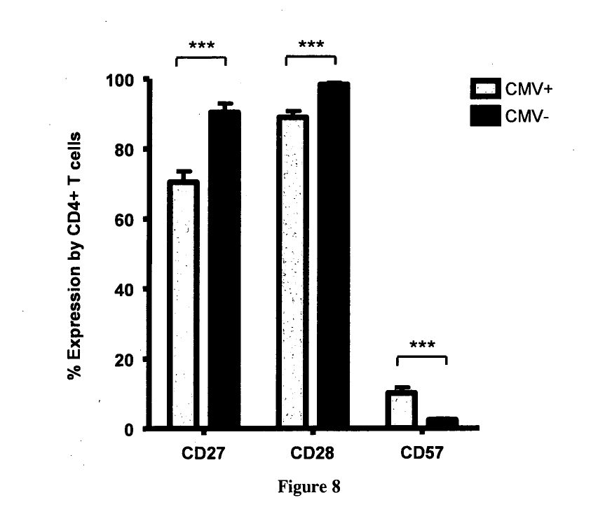

FIG. 8 shows a comparison of CD4+ T cell profiles between healthy donors who are CMV seropositive, and those who are seronegative;

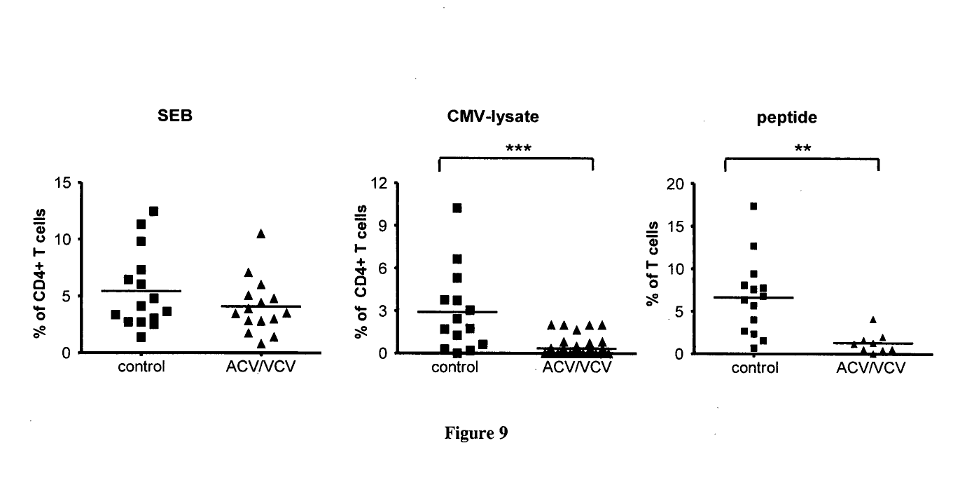

FIG. 9 shows a comparison of CMV-specific T cell counts between patients given acyclovir (ACV) or valacyclovir (VCV), and a control group;

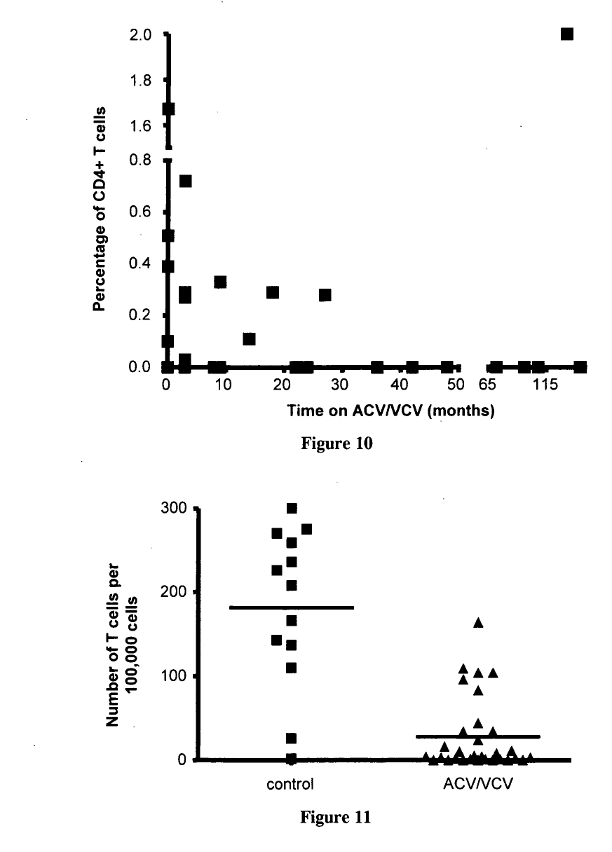

FIG. 10 shows the time-dependent effect of treatment with acyclovir or valacyclovir on the CD4+ T cell response to CMV;

FIG. 11 shows a comparison of the number of antigen-specific T cells as measured by Elispot analysis, between patients given acyclovir or valacyclovir, and a control group;

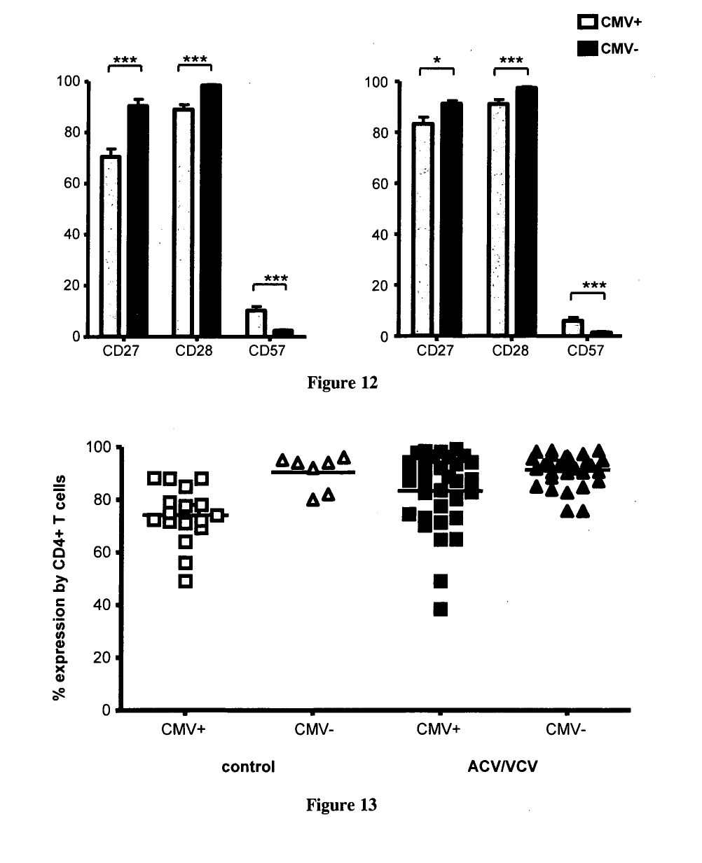

FIG. 12 shows a comparison of the proportions of CD27−, CD28−, or CD57+ CD4+ T cells between patients given acyclovir or valacyclovir, and a control group;

FIG. 13 shows a comparison of the proportion of CD27− CD4+ T cells between patients given acyclovir or valacyclovir, and a control group;

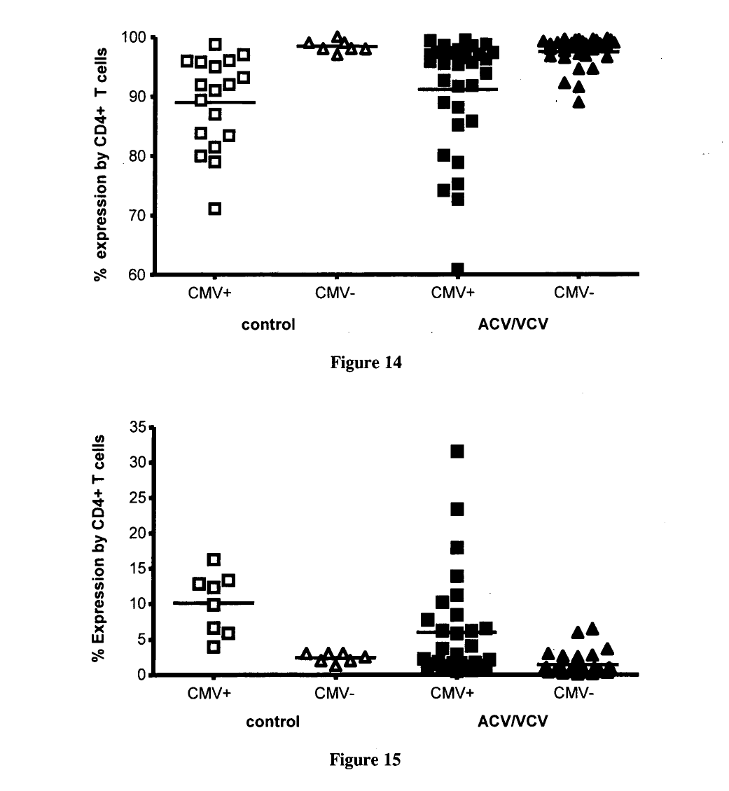

FIG. 14 shows a comparison of the proportion of CD28− CD4+ T cells between patients given acyclovir or valacyclovir, and a control group;

FIG. 15 shows a comparison of the proportion of CD57+ CD4+ T cells between patients given acyclovir or valacyclovir, and a control group;

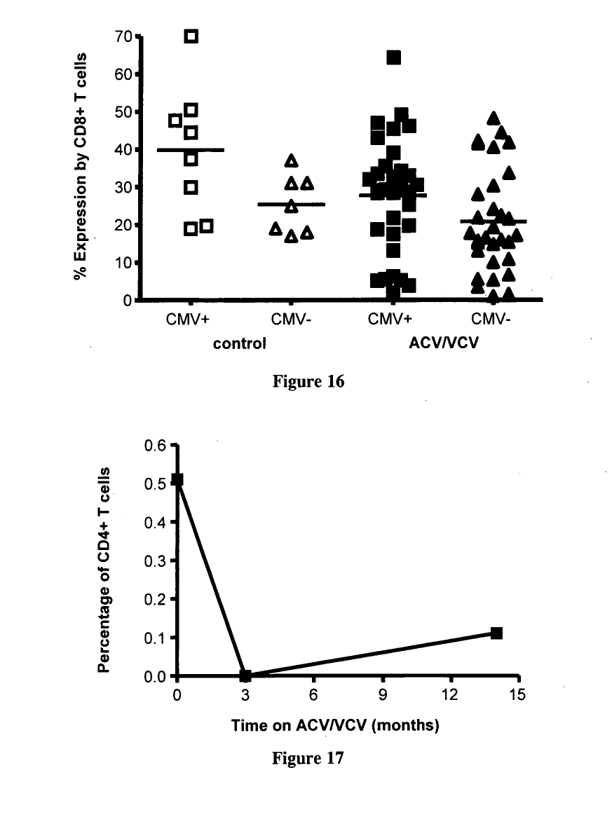

FIG. 16 shows a comparison of the proportion of CD57+ CD8+ T cells between patients given acyclovir or valacyclovir, and a control group;

FIG. 17 shows measurements of the proportion of CMV-specific CD4+ T cells, as measured by stimulation with CMV lysate, in one patient following treatment with acyclovir;

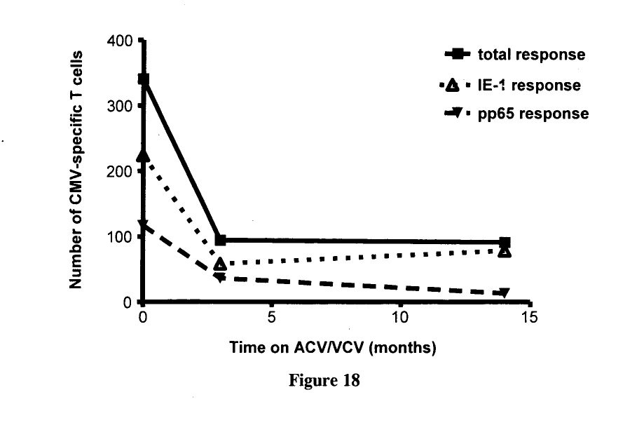

FIG. 18 shows measurements of the proportion of CMV-specific CD4+ T cells, as measured by Elispot analysis, in one patient following treatment with acyclovir; and

Referring to FIG. 1, it can be seen that infection with CMV leads to increased T cell counts in all ages. T cell counts were taken by determination of the absolute lymphocyte count within a peripheral blood sample using an automated cell (‘Coulter’) counter and fluorescent cell (FACS) analysis to determine the percentage of T cells within the lymphoid subset.

Referring to FIG. 2, it can be seen that the increase in T cells is reflected largely in an increase in T cells having the CD8 co-receptor, responsible for cytotoxic activity. Similarly, it can be seen from FIG. 3 that there is a corresponding large increase in ‘antigen-experienced’ memory and effector CD8 T cells. Finally, it can be seen from FIG. 4 that CMV infection is also associated with a reduction in the number of naïve T cells in peripheral blood.

Furthermore, it can be seen from the absolute values of these cell counts in relation to CMV seropositive individuals that the CMV-specific immune response is unusually high in comparison to that associated with other viral infections.

FIG. 5 shows measured steroid profiles (in the form of DHEA/DHEAS ratios) for individuals who test seropositive or seronegative for CMV. It can be seen that CMV infection produces both a greater spread of ratios, and an increase in the average ratio, when compared to the control (CMV seronegative) group. An increased DHEA/DHEAS ratio is typical of inflammatory conditions and is associated with mild immunosuppression.

Referring to FIGS. 6, CMV-specific T cells were detected by expression of Interferon-gamma in response to stimulation with CMV antigen (lysate), and the phenotype of these cells was assessed by staining with antibodies to CD45RA, CD45RO, CD28, CD27 or CD57. It can be seen that CMV-specific CD4+ T cells are characterised by loss of expression of CD27 and/or CD28, and/or gain of expression of CD57. These observations provide an explanation for the influence of CMV infection on CD27, CD28 and CD57 expression on the total CD4+ T cell pool, as documented in FIGS. 8 and 12-15.

Referring to FIG. 7, CMV-specific CD8+ T cells were detected by staining peripheral blood T cells with an HLA-peptide tetramer containing an immunodominant peptide from CMV. Positive staining with the tetramer is shown on the y-axis, and the phenotype of these cells is further characterised by staining with antibodies to CD27, CD28, CD57, CD45RA or CD45RO as shown on the x-axis. It can again be seen that the CMV-specific CD8+ T cells show mainly the CD27−, CD28− and CD57+ phenotypes. These observations provide an explanation for the influence of CMV infection on CD27, CD28 and CD57 expression on the total CD8+ T cell pool as documented in FIG. 16.

Referring to FIG. 8, CD4+ T cells from peripheral blood of 35 healthy donors were stained with antibodies to CD27, CD28 or CD57. It can be seen that the percentages of CD4+ T cells which express these markers are different in CMV-seropositive (n=24) and seronegative (n=11) individuals. The seropositive individuals have greater proportions of CD27−, CD28− and CD57+ cells in the CD4+ populations, compared to seronegative individuals. A increase in CD4+ CD28− T cells to above approximately 2% of the total CD4+ T cell population is seen virtually exclusively in CMV-seropositive individuals.

### EXAMPLE 1

The CMV-specific immune response was measured in a cohort of CMV seropositive or seronegative donors who are taking acyclovir or valaciclovir. This was compared to an age-matched control cohort. The doses given to these donors were typically 400 mg b.d. of acyclovir or 500 mg o.d. or b.d. of valacyclovir. Comprehensive analysis of (1) the total T cell profile of these donors, and (2) their CMV-specific response, was performed. The results were compared to an age-matched control group of CMV seropositive or seronegative donors. The results are shown in FIGS. 9 to 16.

Referring to FIG. 9, blood was obtained from healthy control CMV seropositive donors (n=14) or CMV seropositive patients (n=15) who were taking acyclovir (ACV 400 mg b.d.) or valacyclovir (VCV 500 mg o.d. or b.d.) and incubated with staphylococcus enterotoxin B (as a positive control) or CMV lysate or CMV peptide pools from pp65 and IE-1. Antigen-specific T cells were detected by expression of Interferon-gamma. It can be seen that the number of CMV-specific T cells is reduced in patients taking ACV or VCV.

Referring to FIG. 10, blood was obtained from patients who were taking acyclovir (ACV 400 mg b.d.) or valacyclovir (VCV 500 mg o.d. or b.d.) and incubated with CMV lysate. Antigen-specific T cells were detected by expression of Interferon-gamma. It can again be seen that the number of CMV-specific T cells is reduced in patients who are taking ACV or VCV.

Referring to FIG. 11, blood from healthy CMV seropositive donors or patients taking acyclovir or valacyclovir was stimulated with CMV lysate and the number of antigen-specific T cells was measured by Elispot analysis. A reduced Elispot response to CMV is seen in patients taking ACV or VCV.

In FIG. 12, CD4+ T cells in peripheral blood of 35 CMV-seropositive or seronegative healthy donors were stained with antibodies to CD27, CD28 or CD57. These are shown on the left panel. A similar analysis was performed on patients taking acyclovir or valacyclovir (right hand panel). It can be seen that the percentage of CD4 + T cells which have lost expression of CD27 or CD28, or have gained expression of CD57, is lower in CMV-seropositive patients who are taking ACV or VCV in comparison to healthy donors.

Referring to FIG. 13, CD4+ T cells in peripheral blood of 25 healthy donors or patients taking ACV or VCV were stained with antibodies to CD27. It can be seen that the percentage of CD4 + T cells which have lost expression of CD27 is reduced in CMV-seropositive patients who are taking acyclovir or valacyclovir.

Referring to FIG. 14, CD4+ T cells in peripheral blood of 25 healthy donors or patients taking ACV or VCV were stained with antibodies to CD28. It can be seen that the percentage of CD4+ T cells which have lost expression of CD28 is reduced in CMV-seropositive patients who are taking acyclovir or valacyclovir.

Referring to FIG. 15, CD4+ T cells in peripheral blood of 25 healthy donors or patients taking ACV or VCV were stained with antibodies to CD57. It can be seen that the percentage of CD4+ T cells which have gained expression of CD57 is reduced in CMV seropositive patients who are taking acyclovir or valacyclovir.

In FIG. 16, CD8+ T cells in the peripheral blood of healthy donors or patients taking acyclovir or valacyclovir were stained with antibodies to CD57. The percentage of CD8+ T cells which express CD57 in CMV seropositive donors is reduced in patients who are taking acyclovir or valacyclovir compared to healthy controls.

### EXAMPLE 2

Peripheral blood was taken from a single individual before, and at 3 and 14 months after, treatment with acyclovir 40 mg b.d., and analysed for CMV-specific immune response. The results are shown in FIGS. 17 and 18.

Referring to FIG. 17, the blood was stimulated with CMV lysate, and the percentage of CD4+ T cells which produce interferon-gamma in response to antigen measured. It can be seen that this percentage was reduced on treatment.

Finally, referring to FIG. 18, the blood was stimulated with CMV lysate, or peptide pools from CMV pp65 or IE-1, and the number of CMV-specific T cells was detected using interferon-gamma Elispot analysis. It can be seen that the magnitude of this response was reduced by treatment.

1. A method of medical treatment comprising administering a compound effective against cytomegalovirus (CMV) for improving the immune response of a CMV-seropositive, immunocompetent individual.

2. The method of claim 1, wherein improving the immune response comprises reducing the number of CD4+ CD28− T cells as a proportion of the total number of CD4+ T cells.

3. The method of claim 1, wherein improving the immune response comprises reducing the number of CD4+ CD27− T cells as a proportion of the total number of CD4+ T cells.

4. The method of claim 1, wherein improving the immune response comprises reducing the number of CD4+ CD57+ T cells as a proportion of the total number of CD4+ T cells.

5. The method of claim 1, wherein improving the immune response comprises reducing the number of CD8+ CD28− T cells as a proportion of the total number of CD8+ T cells.

6. The method of claim 1, wherein improving the immune response comprises reducing the number of CD8+ CD27− T cells as a proportion of the total number of CD8+ T cells.

7. The method of claim 1, wherein improving the immune response comprises reducing the number of CD8+ CD57+ T cells as a proportion of the total number of CD8+ T cells.

8. The method of any of claims 2 to 7, wherein the reduction is at least 20%.

9. A method of medical treatment comprising administering a compound effective against CMV for the treatment of CMV in an immunocompetent individual.

10. A method of medical treatment comprising administering a compound effective against CMV for amelioration of the symptoms of a condition selected from HIV infection; mood disorders including depression, fatigue or anxiety; schizophrenia; chronic fatigue syndrome; inflammatory conditions such as rheumatoid arthritis, systemic lupus erythematosus, sarcoidosis, ankylosing spondylitis, psoriatic arthropathy, Wegener's granulomatosis, and other vasculitidies; autoimmune conditions such as multiple sclerosis, Sjorgen's syndrome, diabetes, primary biliary sclerosis, and systemic sclerosis; malignant disease; sarcoidosis or amyloidosis; chronic bacterial, viral or parasitic infection including viral hepatitis and Chagas' disease; malaria; chronic lung disease such as bronchiectasis or cystic fibrosis; acute coronary syndromes such as unstable angina; arterial (e.g. aortic) aneurysm; atherosclerosis; and cerebrovascular occlusion (‘stroke’).

11. (canceled)

12. The method of any of claim 1 to 7, 9 or 10 wherein the compound is selected from acyclovir, famciclovir and valacyclovir.

13. A method of medical treatment comprising administering a nucleoside analogue having an ID₅₀ against CMV of greater than 20 μM for the treatment of CMV-mediated disorders in immunocompetent patients.

14. The method of claim 13, wherein the nucleoside analogue has an ID₅₀ against CMV of at least 50 μM.

15. The method of claim 13, wherein the nucleoside analogue is selected from acyclovir, famciclovir and valacyclovir.
